(19) United States
(12) Patent Application Publication
(10) Pub. No.: US 2025/0134943 A1
(43) Pub. Date: May 1, 2025

(54) HERBAL SUPPLEMENTS AND METHODS OF USE AND MAKING THEREOF

(71) Applicant: KBS RESEARCH, LLC, Dallas, TX (US)

(72) Inventor: Kenneth Brown, Plano, TX (US)

(21) Appl. No.: 18/911,825

(22) Filed: Oct. 10, 2024

Related U.S. Application Data
(60) Provisional application No. 63/593,045, filed on Oct. 25, 2023.

(51) Int. Cl. A61K 36/288; A61K 31/7048; A61K 36/258; A61K 36/284; A61K 36/484; A61K 36/725; A61K 36/746; A61K 36/752; A61P 1/04

(52) U.S. Cl. CPC A61K 36/288; A61K 31/7048; A61K 36/258; A61K 36/284; A61K 36/484; A61K 36/725; A61K 36/746; A61K 36/752; A61P 1/04

(57) ABSTRACT

The present application provides an herbal supplement and method for alleviation of symptoms of gastroesophageal reflux disease (GERD). The herbal supplement comprises two or more herbal components selected from the group consisting of atractloydes extract, dandelion root extract, noni extract, hesperidin extract and quebracho extract.

### RELATED APPLICATIONS

The present application claims priority of U.S. Provisional Patent Application No. 63/593,045, filed on Oct. 25, 2023, which is incorporated by reference in its entirety.

### FIELD

The present application relates generally to dilatory supplements and, more particulaly, to an herbal supplement for alleviation of symptoms of gastroesophageal reflux disease (GERD).

### BACKGROUND

Gastroesophageal reflux disease (GERD) is a common problem treated by physicians. It is estimated that up to 20% of Americans experience symptomatic GERD weekly and that an even higher percentage of people have heartburn monthly. Although GERD is a common clinical diagnosis, its pathogenesis is quite complex. The main pathophysiology of GERD is reflux of the gastric contents, including gastric acid, followed by esophageal mucosal injury or acid stimulation of the esophageal mucosa, even without injury. However, the pathophysiological features are multifactorial, including components of the stomach, gastroesophageal junction (EGJ), nervous system and the esophagus itself There is evidence that LES abnormalities, such as transient LES relaxations (TLESRs) and reduced LES pressure, delayed gastric emptying, sub-optimal esophageal clearance and impaired esophageal mucosal defense, all can contribute.

The current FDA approved medications for gastroesophageal reflux disease are a class called proton pump inhibitors. These drugs have been implicated in certain diseases such as dementia, osteoporosis, stroke, myocardial infarctions and nutrient deficiencies. Natural remedies aiming to help alleviate the symptoms of GERD are in demand and may help prevent chronic consequences such as esophageal cancer without the side effects of pharmacologic options.

### SUMMARY

One aspect of the present application relates to an herbal supplement, comprising two or more herbal components selected from the group consisting of atractloydes extract, dandelion root extract, noni extract, hesperidin and quebracho extract, wherein the supplement is formulated for oral administration for alleviation of symptoms of gastroesophageal reflux disease.

Another aspect of the present application relates to a method for alleviating symptoms of gastroesophageal reflux disease in a human subject. The method comprises the step of orally administering to the human subject in need thereof an effective amount of the herbal supplement of the present application.

Another aspect of the present application relates to a method for preparing the herbal supplement of the present application. The method comprises the steps of mixing the herbal components to form a mixture; and formulating the mixture into unit doses suitable for oral administration.

### DETAILED DESCRIPTION

While the present disclosure will now be described in detail, and it is done so in connection with the illustrative embodiments, it is not limited by the particular embodiments illustrated in the figures and the appended numbered paragraphs.

### I. Definition

Various terms relating to the herbal supplement of the present application are used herein above and also throughout the specification and claims.

Unless defined otherwise, all technical and scientific terms used herein have the same meanings as commonly understood by one of ordinary skill in the art to which this application belongs. All publications and patents specifically mentioned herein are incorporated by reference in their entirety for all purposes including describing and disclosing the chemicals, instruments, statistical analyses and methodologies which are reported in the publications which might be used in connection with the application. All references cited in this specification are to be taken as indicative of the level of skill in the art. Nothing herein is to be construed as an admission that the application is not entitled to antedate such disclosure by virtue of prior invention.

It must be noted that as used herein and in the appended claims, the singular forms “a,” “an,” and “the” include plural reference unless the context clearly dictates otherwise. Further, the terms “a” (or “an”), “one or more” and “at least one” can be used interchangeably herein. It is also to be noted that the terms “comprising,” “including,” “characterized by” and “having” can be used interchangeably.

Any percentages described herein should be considered as being described on a weight to weight (w/w) basis, unless otherwise specified to the contrary (e.g., mole to mole, weight to volume (w/v), etc.). Any ratios described herein should be considered as being described on a weight-to-weight basis, unless otherwise specified to the contrary (e.g., mole to mole, volume to volume, etc.).

As used herein, the tern1 “herbal composition” refers to any phytochemical or mixture thereof that is obtained, isolated, and/or derived from one or more plant materials or plant extracts.

The term “plant material” refers to any plant material including, but not limited to, leaves, hark, stems, flowers, fruits, seeds, roots, and combinations thereof.

The terms “herbal extract,” and “plant extract” are used interchangeably with reference to a composition extracted from a plant. An herbal extract may be in the form of a dry powder, solution or oil (such as essential oils). An herbal extract may contain a mixture of components form the original plant material. An herbal extract may also be in a partially purified form enriched for certain component or components in the original plant.

As used herein, the term “extract ratio” refers to a weight ratio betv1een the raw material and the final extract. The higher is the extract ratio, the more concentrated is the extract. For example, an extract with a 10:1 extract ratio means that 10 parts of the raw material were used to produce 1 part of the extract.

As used herein, the term “substantially free” when used to describe the absence of hydrolysable tannins in a quebracho extract, means that the constituent is present in the quebracho extract as a contaminant in a trace amount of less than 1% (w/w) or 0.1% (w/w).

As used herein, the phrase “treating” or “treatment” of GERD is synonymous with alleviating symptoms of GERD.

The term “nutraceutically acceptable,” such as in the recitation of a “nutraceutically acceptable carrier” refers to a material that is not biologically or otherwise
undesirable, i.e., the material may be incorporated into a herbal composition administered to a patient without causing any undesirable biological effects or interacting in a deleterious manner with any of the other components of the composition in which it is contained.

An “effective amount” refers to a nontoxic but sufficient amount of a composition or plant material to provide a desired systemic or local effect The effective amount will vary with the nature of the composition and constituent parts, the age and physical condition of the end user, the severity of GERD, the duration of the treatment the nature of concurrent therapy, the particular nutraceutically acceptable carrier utilized, and like factors. As used herein, all percentages are by weight unless otherwise specified.

As used herein the transitional term “comprising” and “comprises” are synonymous with “including,” “containing,” or “characterized by,” any one of which is inclusive or open-ended and does not exclude additional, unrecited elements or method steps, regardless of its use in the preamble or the body of a claim. The term further encompasses the terms “consisting of” and “consisting essentially of”. In the claims and/or the specification, “comprising” may mean “one,” but it is also consistent with the meaning of “one or more,” “at least one,” and “one or more than one.”

### II. Compositions

One aspect of the present application relates to an herbal supplement,

comprising two or more herbal components selected from the group consisting of atractloydes extract, dandelion root extract, noni extract, hesperidin extract and quebracho extract, wherein the supplement is formulated for oral administration for alleviation of symptoms of gastroesophageal reflux disease.

### Atractylodes Extract

Atractylodes is a flowering plant in the family Asteraceae. In some embodiments, atractylodes extract is extracted from the rhizome (roots) of certain species of Atractylodes such as Atractylodes lancea, Atractylodes japonica and Atractylodes macrocephala. In some embodiments, the Atractylodes extract is prepared from the rhizome (roots) of Atractylodes lancea, Atractylodes japonica or Atractylodes macrocephala.

In some embodiments, the Atractylodes extracts may comprise at least 0.1%, 0.2%, 0.5%, 1%, 2%, 5%, 10%, 15%, 20% (w/w) or more atractylodin. In some embodiments, the Atractylodes extracts comprise 0.1-20%, 0.1-15%, 0.1-10%, 0.1-5%, 0.1-2%, 0.1-1%, 0.1-0.5%, 0.2-20%, 0.2-15%, 0.2-10%, 0.2-5%, 0.2-2%, 0.2-1%, 0.2-0.5%, 0.5-20%, 0.5-15%, 0.5-10%, 0.5-5%, 0.5-2%, 0.5-1%, 1-20%, 1-15%, 1-10%, 1-5%, 1-2%, 2-20%, 2-15%, 2-10%, 2-5%, 5-20%, 5-15%, 5-10%, 10-20%, 10-15%, or 15-20% (w/w) atractylodin. In some embodiments, the Atractylodes extract is in the form of a powder. In some embodiments, the Atractylodes extract has an extract ratio of 1:1, 2:1, 3:1, 4:1, 5:1, 6:1, 7:1, 8:1, 9:1, 10:1, 15:1 or 20:1. In some embodiments, the Atractylodes extract is an Atractylodes macrocephala extract with an extract ratio of 10:1. In some embodiments, the Atractylodes extract is an Atractylodes lancea extract with an extract ratio of 1:1.

In some embodiments, the Atractylodes extract is present in the herbal supplement of the present application in an amount in the range of 10-2500 mg, 10-2000 mg, 10-1500 mg, 10-1250 mg, 10-1000 mg, 10-750 mg, 10-500 mg, 10-250 mg, 10-100 mg, 50-2500 mg, 50-2000 mg, 50-1500 mg, 50-1250 mg, 50-1000 mg, 50-750 mg, 50-500 mg, 50-250 mg, 100-2500 mg, 100-2000 mg, 100-1500 mg, 100-1250 mg, 100-1000 mg, 100-750 mg, 100-500 mg, 100-250 mg, 250-2500 mg, 250-2000 mg, 250-1500 mg, 250-1250 mg, 250-1000 mg, 250-750 mg, or 250-500 mg per unit dose. In some embodiments, the Atractylodes extract is an Atractylodes macrocephala extract with an extract ratio of 10:1 and is present in the herbal supplement of the present application in an amount of 225 mg per unit dose. In some embodiments, the Atractylodes extract is an Atractylodes lancea extract with an extract ratio of 1:1 and is present in the herbal supplement of the present application in an amount of 250 mg per unit dose.

### Dandelion Root Extract

Dandelion is common name for certain species of Taraxacum, a large genus of flowering plants in the family Asteraceae. Dandelion root extract is prepared from the roots of dandelion plant, including but not limited to, Taraxacum officinale (the common dandelion) and Taraxacum erythrospermum (the red-seeded dandelion).

In some embodiments, dandelion root extracts comprise at least 1%, 2%, 5%, 10%, 15%, 20%, 25%, or 30% (w/w) or more taraxasterol. In some embodiments, dandelion root extracts comprise 1-30%, 1-25%, 1-20%, 1-15%, 1-10%, 1-5%, 1-2%, 2-30%, 2-25%, 2-20%, 2-15%, 2-10%, 2-5%, 5-30%, 5-25%, 5-20%, 5-15%, 5-10%, 10-30%, 10-25%, 10-20%, 10-15%, 15-30%, 15-25%, 15-20%, 20-30%, 20-25%, or 25-30% (w/w) taraxasterol. In some embodiments, dandelion root extract is in the form of a powder. In some embodiments, dandelion root extract has an extract ratio of 1:1, 2:1, 3:1, 4:1, 5:1, 6:1, 7:1, 8:1, 9:1, 10:1, 15:1 or 20:1. In some embodiments, dandelion root extract has an extract ratio of 10:1. In some embodiments, dandelion root extract has an extract ratio of 5:1.

In some embodiments, dandelion root extract is present in the herbal supplement of the present application in an amount in the range of 50-7500 mg, 50-6000 mg, 50-4500 mg, 50-3000 mg, 50-2000 mg, 50-1000 mg, 50-500 mg, 50-250 mg, 50-150 mg, 100-7500 mg, 100-6000 mg, 100-4500 mg, 100-3000 mg, 100-2000 mg, 100-1000 mg, 100-500 mg, 100-250 mg, 100-150 mg, 150-7500 mg, 150-6000 mg, 150-4500 mg, 150-3000 mg, 150-2000 mg, 150-1000 mg, 150-500 mg, 500-7500 mg, 500-6000 mg, 500-4500 mg, 500-3000 mg, 500-2000 mg, 500-1000 mg, 1000-7500 mg, 1000-6000 mg, 1000-4500 mg, 1000-3000 mg, 1000-2000 mg, 2000-7500 mg, 2000-6000 mg, 2000-4500 mg, 2000-3000 mg, 3000-7500 mg, 3000-6000 mg, or 3000-4500 mg per unit dose. In some embodiments, dandelion root extract has an extract ratio of 10:1 and is present in the herbal supplement of the present application in an amount of 125 mg per unit dose. In some embodiments, dandelion root extract has an extract ratio of 5:1 and is present in the herbal supplement of the present application in an amount of 1575 mg per unit dose.

### Noni Extract

Noni, or Morinda citrofolia, is a small evergreen tree found in the Pacific Islands, Southeast Asia, Australia, and India. In some embodiments, noni extract is prepared from the fruit of noni. In some embodiments, noni extract is in the form of a powder. In some embodiments, noni extract has an extract ratio of 1:1, 2:1, 3:1, 4:1, 5:1, 6:1, 7:1, 8:1, 9:1, 10:1, 15:1 or 20:1. In some embodiments, noni extract has an extract ratio of 4:1. In some embodiments, noni extract has an extract ratio of 5:1.

In some embodiments, noni extract is present in the herbal supplement of the present application in an amount in the range of 50-5000 mg, 50-3000 mg, 50-1000 mg, 50-500 mg, 50-150 mg, 150-5000 mg, 150-3000 mg, 150-1000 mg, 150-500 mg, 500-5000 mg, 500-3000 mg, 500-1000 mg, 1500-5000 mg, 1500-3000 mg, or 3000-5000 mg per unit dose. In some embodiments, noni extract has an extract ratio of 5:1 and is present in the herbal supplement of the present application in an amount of 200 mg per unit dose. In some embodiments, noni extract has an extract ratio of 4:1 and is present in the herbal supplement of the present application in an amount of 500 mg per unit dose.

### Hesperidin Extract

Hesperidin belongs to the flavanones subclass of flavonoids and is found primarily in citrus fruits, such as oranges, grapefruits, lemons, and tangerines. In some embodiments, hesperidine extract has a purity of, or has a purity greater than, 80%, 85%, 90% or 95% by HPLC analysis. As used hereinafter, the term “a purity of XX % HPLC” refers to a purity of XX % by HPLC analysis. In some embodiments, hesperidine extract is present in the herbal supplement of the present application in an amount in the range of 10-1000 mg, 10-300 mg, 10-100 mg, 10-30 mg, 30-1000 mg, 30-300 mg, 30-100 mg, 100-1000 mg, 100-300 mg, or 300-1000 mg per unit dose. In some embodiments, hesperidin extract is a citrus aurantium fruit extract with a purity of 90% by HPLC analysis and is present in the herbal supplement of the present application in an amount of 50 mg per unit dose. In some embodiments, hesperidin extract is present in the herbal supplement of the present application in an amount of 100 mg per unit dose.

### Quebracho Extract

The quebracho extract may be derived from any quebracho tree, including but not limited to Shinopsis lorentzii, Schinopsis balansae, Schinopsis brasiliensis, Schinopsis haenkeana, Schinopsis heterophylla and Schinopsis marginata.

In some embodiments, the quebracho extract is prepared from the heartwood of red quebracho trees. In some embodiments, the quebracho extract is substantially free of hydrolyzable tannins. In some embodiments, the quebracho extract is in the form of a powder. In some embodiments, the quebracho extract is in the form of a powder with a condensed tannin content of at least 50%, 55%, 60%, 65%, 70%, 75%, 80%, 85% or 90% (w/w). In some embodiments, the quebracho extract is substantially free of hydrolysable tannins and has a condensed tannin content of at least 50%, at least 60%, or at least 70%. In some embodiments, the quebracho extract is substantially free of hydrolysable tannins and has a condensed tannin content of about 73%. In some embodiments, the quebracho extract has an extract ratio of 1:1, 2:1, 3:1, 4:1, 5:1, 6:1, 7:1, 8:1, 9:1, 10:1, 15:1 or 20:1.

In some embodiments, the quebracho extract is present in the herbal supplement of the present application in an amount in the range of 25-2500 mg, 25-2000 mg, 25-1500 mg, 25-1250 mg, 25-1000 mg, 25-750 mg, 25-500 mg, 25-250 mg, 25-100 mg, 50-2500 mg, 50-2000 mg, 50-1500 mg, 50-1250 mg, 50-1000 mg, 50-750 mg, 50-500 mg, 50-250 mg, 100-2500 mg, 100-2000 mg, 100-1500 mg, 100-1250 mg, 100-1000 mg, 100-750 mg, 100-500 mg, 100-250 mg, 250-2500 mg, 250-2000 mg, 250-1500 mg, 250-1250 mg, 250-1000 mg, 250-750 mg, 250-500 mg, 500-2500 mg, 500-2000 mg, 500-1500 mg, 500-1250 mg, 500-750 mg, 1000-2500 mg, 1000-2000 mg, or 1000-1500 mg per unit dose. In some embodiments, the quebracho extract is present in the herbal supplement of the present application in an amount of 250 mg per unit dose.

### Weight Ratio of Components

In some embodiments, the herbal supplement comprises noni extract and hesperidine at a weight ratio in the range of 40:1 to 2:5, 10:1 to 1:2, 5:1 to 1:1, or 5:1 to 3:1. In some embodiments, the herbal supplement comprises noni extract (5:1 extract ratio) and hesperidine (90% HPLC) at a weight ratio of 5:1.

In some embodiments, the herbal supplement comprises noni extract and atractloydes extract at a weight ratio in the range of 10:1 to 1:10, 5:1 to 1:5, 4:1 to 1:4, 3:1 to 1:3, or 2:1 to 1:2. In some embodiments, the herbal supplement comprises noni extract (5:1 extract ratio) and atractloydes extract (10:1 extract ratio) at a weight ratio of 8:9.

In some embodiments, the herbal supplement comprises noni extract and dandelion root extract at a weight ratio in the range of 16:1 to 4:25, 4:1 to 1:5 or 2:1 to 1:2. In some embodiments, the herbal supplement comprises noni extract (5:1 extract ratio) and dandelion root extract (10:1 extract ratio) at a weight ratio of about 8:5.

In some embodiments, the herbal supplement comprises hesperidine and atractloydes extract at a weight ratio in the range of 20:9 to 1:45, 10:9 to 1:25, 5:9 to 1:10, or 1:3 to 1:9. In some embodiments, the herbal supplement comprises hesperidine (90% HPLC) and atractloydes extract (10:1 extract ratio) at a weight ratio of 2:9.

In some embodiments, the herbal supplement comprises hesperidine and dandeline root extract at a weight ratio in the range of 4:1 to 1:25, 2:1 to 1:10, or 1:1 to 1:5. In some embodiments, the herbal supplement comprises hesperidine (90% HPLC) and dandeline root extract (10:1 extract ratio) at a weight ratio of 2:5.

In some embodiments, the herbal supplement comprises atractloydes extract and dandelion root extract at a weight ratio in the range of 18:1 to 9:50, 9:1 to 9:25, or 3:1 to 9:10. In some embodiments, the herbal supplement comprises atractloydes extract (10:1 extract ratio) and dandelion root extract (10:1 extract ratio) at a weight ratio of 9:5.

In some embodiments, the herbal supplement comprises quebracho extract and atractloydes extract at a weight ratio in the range of 10:1 to 1:10, 5:1 to 1:5, 4:1 to 1:4, 3:1 to 1:3, or 2:1 to 1:2. In some embodiments, the herbal supplement comprises quebracho extract and atractloydes extract at a weight ratio of about 1:1.

In some embodiments, the herbal supplement comprises quebracho extract and dandelion root extract at a weight ratio in the range of 5:3 to 1:60, 5:6 to 1:30, 1:2 to 1:15, or 1:3 to 1:10. In some embodiments, the herbal supplement comprises red quebracho extract and dandelion root extract at a weight ratio of about 1:6.

In some embodiments, the herbal supplement comprises quebracho extract and noni extract at a weight ratio in the range of 5:1 to 1:20, 5:2 to 1:10, 2:1 to 1:5, or 1:1 to 1:3. In some embodiments, the herbal supplement comprises red quebracho extract and noni extract at a weight ratio of about 1:2.

In some embodiments, the herbal supplement comprises quebracho extract and hesperidine extract at a weight ratio in the range of 25:1 to 1:4, 10:1 to 1:2, 5:1 to 1:1, or 5:1 to 5:3.

In some embodiments, the herbal supplement comprises quebracho extract and hesperidine extract at a weight ratio of about 5:2.

In some embodiments, the herbal supplement comprises quebracho extract, atractloydes extract and dandrilion root extract at a weight ratio in the range of 1:1:6.

In some embodiments, the herbal supplement comprises quebracho extract, atractloydes extract and noni extract at a weight ratio in the range of 1:1:2.

In some embodiments, the herbal supplement comprises quebracho extract, atractloydes extract and hesperidine extract at a weight ratio in the range of 5:5:2.

In some embodiments, the herbal supplement comprises quebracho extract, dandelion root extract and noni extract a weight ratio in the range of 1:6:2.

In some embodiments, the herbal supplement comprises quebracho extract, dandelion root extract and hesperidine extract a weight ratio in the range of 5:30:2.

In some embodiments, the herbal supplement comprises quebracho extract, noni extract and hesperidine extract a weight ratio in the range of 5:10:2.

In some embodiments, the herbal supplement comprises atractloydes extract, dandelion root extract and noni extract at a weight ratio in the range of 1:6:2. In some embodiments, the herbal supplement comprises atractloydes extract (10:1 extract ratio), dandelion root extract (10:1 extract ratio) and noni extract (5:1 extract ratio) at a weight ratio in the range of 9:5:8.

In some embodiments, the herbal supplement comprises atractloydes extract, dandelion root extract and hesperidine extract at a weight ratio in the range of 5:30:2. In some embodiments, the herbal supplement comprises atractloydes extract (10:1 extract ratio), dandelion root extract (10:1 extract ratio) and hesperidin extract (90% HPLC) at a weight ratio in the range of 9:5:2.

In some embodiments, the herbal supplement comprises dandelion root extract, noni extract and hesperidine extract at a weight ratio in the range of 15:5:1. In some embodiments, the herbal supplement comprises dandelion root extract (10:1 extract ratio), noni extract (5:1 extract ratio) and hesperidin extract (90% HPLC) at a weight ratio in the range of 5:8:2.

In some embodiments, the herbal supplement comprises red quebracho extract, atractloydes extract, dandelion root extract and noni extract at a weight ratio in the range of 1:1:6:2.

In some embodiments, the herbal supplement comprises red quebracho extract, atractloydes extract, dandelion root extract and hesperidine extract at a weight ratio in the range of 5:5:30:2.

In some embodiments, the herbal supplement comprises red quebracho extract, atractloydes extract, noni extract and hesperidine extract at a weight ratio in the range of 5:5:10:2.

In some embodiments, the herbal supplement comprises red quebracho extract, dandelion root extract, noni extract and hesperidine extract at a weight ratio in the range of 5:30:10:2.

In some embodiments, the herbal supplement comprises atractloydes extract, dandelion root extract, noni extract and hesperidine extract at a weight ratio in the range of 5:30:10:2.

In some embodiments, the herbal supplement comprises atractloydes extract (10:1 extraction ratio), dandelion root extract (10:1 extraction ratio), noni extract (5:1 extraction ratio) and hesperidine extract (90% HPLC) at a weight ratio in the range of 9:5:8:2.

In some embodiments, the herbal supplement comprises red quebracho extract, atractloydes extract, dandelion root extract, noni extract and hesperidine extract at a weight ratio in the range of 5:5:30:10:2.

In some embodiments, the herbal supplement comprises 1-25 weight parts of red quebracho extract, 1-25 weight parts of atractloydes extract and 6-150 weight parts of dandelion root extract. In some embodiments, the herbal supplement comprises 2-15 weight parts of red quebracho extract, 2-15 weight parts of atractloydes extract and 10-90 weight parts of dandelion root extract. In some embodiments, the herbal supplement comprises 4-10 weight parts of red quebracho extract, 4-10 weight parts of atractloydes extract and 20-60 weight parts of dandelion root extract. As used herein, the term “weight part” refers to a weight unit relative to the weight of another component in the same composition. For example, an herbal composition comprising 1 gram of red quebracho extract, 1 gram of atractloydes extract and 6 gram of dandelion root extract can be described as having 1 weight part of red quebracho extract, 1 weight part of atractloydes extract and 6 weight part of dandelion root extract.

In some embodiments, the herbal supplement comprises 1-25 weight parts of red quebracho extract, 1-25 weight parts of atractloydes extract and 2-50 weight parts of noni extract. In some embodiments, the herbal supplement comprises 2-15 weight parts of red quebracho extract, 2-15 weight parts of atractloydes extract and 4-30 weight parts of noni extract. In some embodiments, the herbal supplement comprises 4-10 weight parts of red quebracho extract, 4-10 weight parts of atractloydes extract and 8-20 weight parts of noni extract.

In some embodiments, the herbal supplement comprises 1-25 weight parts of red quebracho extract, 1-25 weight parts of atractloydes extract and 0.4-10 weight parts of hesperidine extract. In some embodiments, the herbal supplement comprises 2-15 weight parts of red quebracho extract, 2-15 weight parts of atractloydes extract and 0.8-6 weight parts of hesperidine extract. In some embodiments, the herbal supplement comprises 4-10 weight parts of red quebracho extract, 4-10 weight parts of atractloydes extract and 1.6-4 weight parts of hesperidine extract.

In some embodiments, the herbal supplement comprises 1-25 weight parts of red quebracho extract, 6-150 weight parts of dandelion root extract and 2-50 weight parts of noni extract. In some embodiments, the herbal supplement comprises 2-15 weight parts of red quebracho extract, 12-90 weight parts of dandelion root extract and 4-30 weight parts of noni extract. In some embodiments, the herbal supplement comprises 4-10 weight parts of red quebracho extract, 24-60 weight parts of dandelion root extract and 8-20 weight parts of noni extract.

In some embodiments, the herbal supplement comprises 1-25 weight parts of red quebracho extract, 6-150 weight parts of dandelion root extract and 0.4-10 weight parts of hesperidine extract. In some embodiments, the herbal supplement comprises 2-15 weight parts of red quebracho extract, 12-90 weight parts of dandelion root extract and 0.8-6 weight parts of hesperidine extract. In some embodiments, the herbal supplement comprises 4-10 weight parts of red quebracho extract, 24-60 weight parts of dandelion root extract and 1.6-4 weight parts of hesperidine extract.

In some embodiments, the herbal supplement comprises 1-25 weight parts of red quebracho extract, 2-50 weight parts of noni extract and 0.4-10 weight parts of hesperidine extract. In some embodiments, the herbal supplement comprises 2-15 weight parts of red quebracho extract, 4-30 weight parts of noni extract and 0.8-6 weight parts of hesperidine extract. In some embodiments, the herbal supplement comprises 4-10 weight parts of red quebracho extract, 8-20 weight parts of noni extract and 1.6-4 weight parts of hesperidine extract.

In some embodiments, the herbal supplement comprises 1-25 weight parts of atractloydes extract, 6-150 weight parts of dandelion root extract and 2-50 weight parts of noni extract. In some embodiments, the herbal supplement comprises 2-15 weight parts of atractloydes extract, 12-90 weight parts of dandelion root extract and 4-30 weight parts of noni extract. In some embodiments, the herbal supplement comprises 4-10 weight parts of atractloydes extract, 24-60 weight parts of dandelion root extract and 8-20 weight parts of noni extract.

In some embodiments, the herbal supplement comprises 1-25 weight parts of atractloydes extract, 6-150 weight parts of dandelion root extract and 0.4-10 weight parts of hesperidine extract. In some embodiments, the herbal supplement comprises 2-15 weight parts of atractloydes extract, 12-90 weight parts of dandelion root extract and 0.8-6 weight parts of hesperidine extract. In some embodiments, the herbal supplement comprises 4-10 weight parts of atractloydes extract, 24-60 weight parts of dandelion root extract and 1.6-4 weight parts of hesperidine extract.

In some embodiments, the herbal supplement comprises 6-150 weight parts of dandelion root extract, 2-50 weight parts of noni extract and 0.4-10 weight parts of hesperidine extract. In some embodiments, the herbal supplement comprises 12-90 weight parts of dandelion root extract, 4-30 weight parts of noni extract and 0.8-6 weight parts of hesperidine extract. In some embodiments, the herbal supplement comprises 24-60 weight parts of dandelion root extract, 8-20 weight parts of noni extract and 1.6-4 weight parts of hesperidine extract.

In some embodiments, the herbal supplement comprises 1-25 weight parts of red quebracho extract, 1-25 weight parts of atractloydes extract, 6-150 weight parts of dandelion root extract and 2-50 weight parts of noni extract. In some embodiments, the herbal supplement comprises 2-15 weight parts of red quebracho extract, 2-15 weight parts of atractloydes extract 10-90 weight parts of dandelion root extract and 4-30 weight parts of noni extract. In some embodiments, the herbal supplement comprises 4-10 weight parts of red quebracho extract, 4-10 weight parts of atractloydes extract, 20-60 weight parts of dandelion root extract and 8-20 weight parts of noni extract.

In some embodiments, the herbal supplement comprises red quebracho extract, atractloydes extract, dandelion root extract and hesperidine extract at a weight ratio in the range of 5:5:30:2.

In some embodiments, the herbal supplement comprises 1-25 weight parts of red quebracho extract, 1-25 weight parts of atractloydes extract, 6-150 weight parts of dandelion root extract and 0.4-10 weight parts of hesperidine extract. In some embodiments, the herbal supplement comprises 2-15 weight parts of red quebracho extract, 2-15 weight parts of atractloydes extract 10-90 weight parts of dandelion root extract and 0.8-6 weight parts of hesperidine extract. In some embodiments, the herbal supplement comprises 4-10 weight parts of red quebracho extract, 4-10 weight parts of atractloydes extract, 20-60 weight parts of dandelion root extract and 1.6-4 weight parts of hesperidine extract.

In some embodiments, the herbal supplement comprises 1-25 weight parts of red quebracho extract, 1-25 weight parts of atractloydes extract, 2-50 weight parts of noni extract and 0.4-10 weight parts of hesperidine extract. In some embodiments, the herbal supplement comprises 2-15 weight parts of red quebracho extract, 2-15 weight parts of atractloydes extract 4-30 weight parts of noni extract and 0.8-6 weight parts of hesperidine extract. In some embodiments, the herbal supplement comprises 4-10 weight parts of red quebracho extract, 4-10 weight parts of atractloydes extract, 8-20 weight parts of noni extract and 1.6-4 weight parts of hesperidine extract.

In some embodiments, the herbal supplement comprises 1-25 weight parts of red quebracho extract, 6-150 weight parts of dandelion root extract, 2-50 weight parts of noni extract and 0.4-10 weight parts of hesperidine extract. In some embodiments, the herbal supplement comprises 2-15 weight parts of red quebracho extract, 12-90 weight parts of dandelion root extract 4-30 weight parts of noni extract and 0.8-6 weight parts of hesperidine extract. In some embodiments, the herbal supplement comprises 4-10 weight parts of red quebracho extract, 24-60 weight parts of dandelion root extract, 8-20 weight parts of noni extract and 1.6-4 weight parts of hesperidine extract.

In some embodiments, the herbal supplement comprises atractloydes extract, dandelion root extract, noni extract and hesperidine extract at a weight ratio in the range of 5:30:10:2.

In some embodiments, the herbal supplement comprises 1-25 weight parts of atractloydes extract, 6-150 weight parts of dandelion root extract, 2-50 weight parts of noni extract and 0.4-10 weight parts of hesperidine extract. In some embodiments, the herbal supplement comprises 2-15 weight parts of atractloydes extract, 12-90 weight parts of dandelion root extract 4-30 weight parts of noni extract and 0.8-6 weight parts of hesperidine extract. In some embodiments, the herbal supplement comprises 4-10 weight parts of atractloydes extract, 24-60 weight parts of dandelion root extract, 8-20 weight parts of noni extract and 1.6-4 weight parts of hesperidine extract.

In some embodiments, the herbal supplement comprises 1-25 weight parts of red quebracho extract, 1-25 weight parts of atractloydes extract, 6-150 weight parts of dandelion root extract, 2-50 weight parts of noni extract and 0.4-10 weight parts of hesperidine extract. In some embodiments, the herbal supplement comprises 2-15 weight parts of red quebracho extract, 2-15 weight parts of atractloydes extract, 12-90 weight parts of dandelion root extract 4-30 weight parts of noni extract and 0.8-6 weight parts of hesperidine extract. In some embodiments, the herbal supplement comprises 4-10 weight parts of red quebracho extract, 4-10 weight parts of atractloydes extract, 24-60 weight parts of dandelion root extract, 8-20 weight parts of noni extract and 1.6-4 weight parts of hesperidine extract.

In some embodiments, the herbal supplement comprises 0.9-90 weight parts of atractloydes extract, 0.5-50 weight parts of dandelion root extract, 0.8-80 weight parts of noni extract and 0.2-20 weight parts of hesperidine extract. In some embodiments, the herbal supplement comprises 3-30 weight parts of atractloydes extract, 2-20 weight parts of dandelion root extract, 4-20 weight parts of noni extract and 0.2-20 weight parts of hesperidine extract. In some embodiments, the herbal supplement comprises 3-15 weight parts of atractloydes extract, 3-8 weight parts of dandelion root extract, 4-12 weight parts of noni extract and 1-3 weight parts of hesperidine extract. In some embodiments, the atractloydes extract has an extraction ratio of 10:1, the dandelion root extract has an extraction ratio of 10:1, the noni extract has an extraction ratio of 5:1 and the hesperidine extract has a purity of 90% HPLC.

In some embodiments, the herbal supplement comprises 0.9-90 weight parts of atractloydes extract, 0.5-50 weight parts of dandelion root extract, and 0.8-80 weight parts of noni extract. In some embodiments, the herbal supplement comprises 3-30 weight parts of atractloydes extract, 2-20 weight parts of dandelion root extract, and 4-20 weight parts of noni extract. In some embodiments, the herbal supplement comprises 3-15 weight parts of atractloydes extract, 3-8 weight parts of dandelion root extract, and 4-12 weight parts of noni extract. In some embodiments, the atractloydes extract has an extraction ratio of 10:1, the dandelion root extract has an extraction ratio of 10:1, and the noni extract has an extraction ratio of 5:1.

In some embodiments, the herbal supplement comprises 0.9-90 weight parts of atractloydes extract, 0.5-50 weight parts of dandelion root extract, and 0.2-20 weight parts of hesperidine extract. In some embodiments, the herbal supplement comprises 3-30 weight parts of atractloydes extract, 2-20 weight parts of dandelion root extract, and 0.2-20 weight parts of hesperidine extract. In some embodiments, the herbal supplement comprises 3-15 weight parts of atractloydes extract, 3-8 weight parts of dandelion root extract, and 1-3 weight parts of hesperidine extract. In some embodiments, the atractloydes extract has an extraction ratio of 10:1, the dandelion root extract has an extraction ratio of 10:1, and the hesperidine extract has a purity of 90% HPLC.

In some embodiments, the herbal supplement comprises 0.9-90 weight parts of atractloydes extract, 0.8-80 weight parts of noni extract and 0.2-20 weight parts of hesperidine extract. In some embodiments, the herbal supplement comprises 3-30 weight parts of atractloydes extract, 4-20 weight parts of noni extract and 0.2-20 weight parts of hesperidine extract. In some embodiments, the herbal supplement comprises 3-15 weight parts of atractloydes extract, 4-12 weight parts of noni extract and 1-3 weight parts of hesperidine extract. In some embodiments, the atractloydes extract has an extraction ratio of 10:1, the noni extract has an extraction ratio of 5:1 and the hesperidine extract has a purity of 90% HPLC.

In some embodiments, the herbal supplement comprises 0.5-50 weight parts of dandelion root extract, 0.8-80 weight parts of noni extract and 0.2-20 weight parts of hesperidine extract. In some embodiments, the herbal supplement comprises 2-20 weight parts of dandelion root extract, 4-20 weight parts of noni extract and 0.2-20 weight parts of hesperidine extract. In some embodiments, the herbal supplement comprises 3-8 weight parts of dandelion root extract, 4-12 weight parts of noni extract and 1-3 weight parts of hesperidine extract. In some embodiments, the dandelion root extract has an extraction ratio of 10:1, the noni extract has an extraction ratio of 5:1 and the hesperidine extract has a purity of 90% HPLC.

In some embodiments, the herbal supplement comprises 0.9-90 weight parts of atractloydes extract and 0.5-50 weight parts of dandelion root extract. In some embodiments, the herbal supplement comprises 3-30 weight parts of atractloydes extract and 2-20 weight parts of dandelion root extract. In some embodiments, the herbal supplement comprises 3-15 weight parts of atractloydes extract and 3-8 weight parts of dandelion root extract. In some embodiments, the atractloydes extract has an extraction ratio of 10:1, and the dandelion root extract has an extraction ratio of 10:1.

In some embodiments, the herbal supplement comprises 0.9-90 weight parts of atractloydes extract and 0.8-80 weight parts of noni extract. In some embodiments, the herbal supplement comprises 3-30 weight parts of atractloydes extract and 4-20 weight parts of noni extract. In some embodiments, the herbal supplement comprises 3-15 weight parts of atractloydes extract and 4-12 weight parts of noni extract. In some embodiments, the atractloydes extract has an extraction ratio of 10:1 and the noni extract has an extraction ratio of 5:1.

In some embodiments, the herbal supplement comprises 0.9-90 weight parts of atractloydes extract and 0.2-20 weight parts of hesperidine extract. In some embodiments, the herbal supplement comprises 3-30 weight parts of atractloydes extract and 0.2-20 weight parts of hesperidine extract. In some embodiments, the herbal supplement comprises 3-15 weight parts of atractloydes extract and 1-3 weight parts of hesperidine extract. In some embodiments, the atractloydes extract has an extraction ratio of 10:1 and the hesperidine extract has a purity of 90% HPLC.

In some embodiments, the herbal supplement comprises 0.5-50 weight parts of dandelion root extract and 0.8-80 weight parts of noni extract. In some embodiments, the herbal supplement comprises 2-20 weight parts of dandelion root extract and 4-20 weight parts of noni extract. In some embodiments, the herbal supplement comprises 3-8 weight parts of dandelion root extract and 4-12 weight parts of noni extract. In some embodiments, the dandelion root extract has an extraction ratio of 10:1 and the noni extract has an extraction ratio of 5:1.

In some embodiments, the herbal supplement comprises 0.5-50 weight parts of dandelion root extract and 0.2-20 weight parts of hesperidine extract. In some embodiments, the herbal supplement comprises 2-20 weight parts of dandelion root extract and 0.2-20 weight parts of hesperidine extract. In some embodiments, the herbal supplement comprises 3-15 weight parts of 3-8 weight parts of dandelion root extract and 1-3 weight parts of hesperidine extract. In some embodiments, the dandelion root extract has an extraction ratio of 10:1 and the hesperidine extract has a purity of 90% HPLC.

In some embodiments, the herbal supplement comprises 0.8-80 weight parts of noni extract and 0.2-20 weight parts of hesperidine extract. In some embodiments, the herbal supplement comprises 4-20 weight parts of noni extract and 0.2-20 weight parts of hesperidine extract. In some embodiments, the herbal supplement comprises 4-12 weight parts of noni extract and 1-3 weight parts of hesperidine extract. In some embodiments, the noni extract has an extraction ratio of 5:1 and the hesperidine extract has a purity of 90% HPLC.

### Additional Components

In some embodiments, the herbal supplement of the present application further comprises additional components selected from the group consisting of citrus unshiu peel extracts, genseng extracts, jujube extracts, deglycyrrhizinated licorice (DGL) and Ceratonia siliqua (Carob) extracts.

In some embodiments, the herbal supplement of the present application further comprises a nutraceutically acceptable carrier.

### III. Method of Making

The herbal supplement of the present application may be prepared by mixing the suitable plant extract(s) and components to form a mixture and then formulating the mixture into the suitable dosage form. In some embodiments, the herbal supplement is in the form of a tablet or capsule.

Extracts for use in the present application may be produced from plant tissues/parts that can be extracted by water, polar, or petroleum solvents for treating irritable bowel syndrome. An extract may be prepared using extraction procedures well known in the art (e.g., the use of organic solvents such as lower alcohols, alkyl esters, alkyl ethers, alkyl ketones, chloroform, petroleum ether, hexane and/or inorganic solvents such as water) Additionally, the extracts may be produced by cold extraction techniques using a variety of different extraction solvents including, bm not limited to, water, fatty solvents (such as olive oil), and alcoholic solvents (e.g. 70 /o ethanol). Cold extraction techniques may be applied to softer parts of the plant such as leaves and flowers, or in cases wherein the desired active components of the plant are heat labile.

Alternatively, the aforementioned solvents may be used to produce extracts of the desired plants by a hot extraction technique, wherein the solvents are heated to a high temperature, the precise value of said temperature being dependent on the properties of the chosen solvent, and maintained at that temperature throughout the extraction process. Hot extraction techniques are more commonly applied to the harder, tougher parts of the plant, such as bark, woody branches and larger roots.

In some cases, sequential extractions may be performed in more than one solvent, and at different temperatures.

Standard procedures for producing plant extracts, including hot extraction, cold extraction and other techniques, are commonly known in the art. Preferably, the supplement comprises one or more plant extract(s) prepared in powdered form.

In certain embodiments, the supplement may include an herbal extract in the form of an essential oil. As used herein, the term “essential oil” refers to an oil derived from herbs or other plants through steam distillation or cold pressing. An essential oil may be mixed with a vegetable oil or water and may be orally ingested for use in accordance with the present disclosures.

In some cases, leaves from a given plant species may be steam distilled to 1nake the essential oil from that plant species. The essential oil made from a defined tree species may be used interchangeably for the name of the plant species and for the name of the essential oil produced from the leaves and other components of the plant species. Likewise, the same applies to an extract derived from a given plant species.

The herbal supplement can be provided in any nutraceutically acceptable form. Preferably, the herbal supplement is formulated for oral administration as, for example but not limited to, drug powders, crystals, granules, small particles (which include particles sized on the order of micrometers, such as microspheres and microcapsules), particles (which include particles sized on the order of millimeters), beads, 1nicrobeads, pellets, pills, microtablets, compressed tablets or tablet triturates, molded tablets or tablet triturates, and in capsules, which are either hard or soft and contain the composition as a powder, panicle, bead, solution or suspension.

The herbal supplement can also be formulated for oral administration as a solution or suspension in an aqueous liquid, as a liquid incorporated into a gel capsule or as any other convenient formulation for administration, or for rectal administration, as a suppository, enema or other convenient folm. In addition, the compositions of the application can also be provided as a controlled release system.

The herbal supplement may further include any nutraceutically acceptable excipient, carrier or mixture thereof. As used herein, the term “nutraceutically acceptable excipient or carrier” refers to a non-toxic, inert solid, semi-solid, diluent, encapsulating material or formulation auxiliary of any type. Exemplary excipients include, but are not limited to diluents or fillers, such as dextrates, dicalcium phosphate, calcium sulfate, lactose, cellulose, kaolin, mannitol, sodium chloride, dry starch, sorbitol, sucrose, inositol, powdered sugar, bentonite, microcrystalline cellulose, or hydroxypropylmethylcellulose may be added to the inhibitor molecule to increase the bulk of the composition. Also, hinders, such as but not limited to, starch, gelatin, sucrose, glucose, dextrose, molasses, lactose, acacia gum, sodium alginate, extract of lrish moss, panwar gum, ghatti gum, mucilage of isapgol husks, carboxymethylcellulose, methylcellulose, polyvinylpyrrolidone, Veegum and starch arabogalactan, polyethylene glycol, ethyl cellulose, and \vaxes, may be added to the supplement to increase its cohesive qualities. Additionally, lubricants, such as but not limited to, talc, magnesium stearate, calcium stearate, stearic acid, hydrogenated vegetable oils, polyethylene glycol, sodium benzoate, sodium acetate, sodium chloride, leucine, carbowax, sodium lauryl sulfate, and magnesium lauryl sulfate may be added to the supplement. Also, glidants, such as but not limited to, colloidal silicon dioxide or talc may be added to improve the flow characteristics of a powdered supplement. Finally, disintegrants, such as but not limited to, starches, clays, celluloses, algins,, gums, crosslinked polymers (e.g., croscarmelose, crospovidone, and sodium starch glycolate), Veegum, methykellulose, agar, bentonite, cellulose and wood products, natural sponge, cation-exchange resins, alginic acid, guar gum, citrus pulp, carboxymethylcellulose, or sodium lauryl sulfate with starch may also be added to facilitate disintegration of the supplement in the intestine.

In certain embodiments, one or more compositions of the supplement may be formulated to protect the composition from degradation by the acidic conditions of the stomach and from interactions with proteins, such as pepsin, present in the stomach. Such a formulation may include a pH-dependent enteric coating to prevent release until after gastric emptying. Thus, in some embodiments, one or more compositions or the entire supplement is enteric coated. However, in other embodiments no compositions of the supplement are enteric coated.

An enteric coated composition or supplement may be formulated as enteric coaled tablets, beads or granules, which may optionally contain a lubricant such as, but not limited to, magnesium stearate. The enteric coating may include one or more pH dependent polymers. The pH dependent polymers may remain intact at pH values lower than about 4.0 and dissolve at pH values higher than 40, preferably higher than 50, most preferably about 6.0. Exemplary pH-dependent polymers include, but are not limited to, methacarylic acid copolymers, methacrylic acid-methyl methacrylate copolymers (e.g., EUDRAGIT® L100 (Type A), EUDRAGIT® S100 (Type B), Rohm GmbH, Germany; methacrylic acid-ethyl acrylate copolymers (e.g., EUDRAGIT® L100-55 (Type C) and EUDRAGIT®L30D-55 copolymer dispersion, Rohm GmbH, Germany), copolymers of methacrylic acid-methyl methacrylate and methyl methacrylate (EUDRAGIT® FS), terpolymers of methacrylic acid, methacrylate, and ethyl acrylate, hydroxypropyl methylcellulose phthalate (HPMCP) (e.g., HP-55, HP-50, HP-55S, Shinetsu Chemical, Japan), polyvinyl acetate phthalates (PVAP) (e.g., COATERIC®, OPADRY® enteric white (OY-P-7171), polyvinylbutyrate acetate; cellulose acetate succinates (CAS); hydroxypropyl methylcellulose acetate succinate (HPMCAS), e.g., HPMCAS LF Grade, MF Grade, HF Grade, including AQOAT®LF and AQOAT®MF (ShinEtsu Chemical, Japan), shellac (e.g., MARCOAT™ 125 & MARCOAT™ 125N), vinyl acetate-maleic anhydride copolymer, styrene-maleic monoester copolymer, carboxymethyl ethylcellulose (CMEC, Freund Corporation, Japan), cellulose acetate phthalates (CAP) (e.g., AQUATERIC®): cellulose acetate trimellitates (CAT) and mixtures of two or more thereof at weight ratios between about 2:1 to about 5:1, such as, for instance, a mixture of EUDRAGIT® L100-55 and EUDRAGIT® S100 at a weight ratio of about 3:1 to about 2:1, or a mixture of EUDRAGIT® L30 D-55 and EUDRAGIT® FS at a weight ratio of about 3:1 to about 5:1.

The pH dependent polymers can be incorporated in an amount from about 10% to 90%, preferably from about 20% to 80% and most preferably from about 30% to 70% by weight of the dosage unit or supplement. The polymer(s) can be incorporated into the formulation either prior to or after granulation or they can be added into the supplement either as a dry material, or they can be dispersed or dissolved in an appropriate solvent, and dispersed during granulation.

An enteric coated composition or supplement may include enteric coated beads in a capsule, enteric coated microspheres in a capsule, enteric coated microspheres provided in a suspension or mixed with food, which are particularly convenient for pediatric administration, and enteric coated compressed tablets. The capsule can be a hard-shell gelatin capsule or a cellulose capsule. In particular, the composition or herbal supplement may be formulated as an enteric coated capsule.

In some embodiments, the composition(s) and/or supplements may be directly compressed, with or without any excipients, into a tablet or other herbal supplement having a nutraceutically acceptable hardness and friability. Preferably, the directly compressible herbal supplement can be compressed into tablets having a hardness of greater than 4 kp (kiloponds), preferably a hardness of 8 to 14 kp, more preferably a hardness of 10 to 13 kp. A directly compressible composition can be corn pressed into a tablet that has a friability of not more than 1% loss in weight, preferably less than 0.8% loss in weight, more preferably less than 0.5% loss in weight.

### IV. Method of Use (Methods of Treatment)

Another aspect of the present application relates to a method of preventing, treating or alleviating a symptom of GERD. The method comprises the step of administering to a subject in need of such treatment, an effective amount of the herbal supplement of the present application. In some embodiments, the subject is a human subject. In some embodiments, the subject has one or more symptoms of GERD. In some embodiments, the subject is diagnosed with GRED. In some embodiments, the subject has been treated with proton pump inhibitors such as omeprazole, lansoprazole, pantoprazole, rabeprazole, esomeprazole and dexlansoprazole, and/or H2 blockers such as Pepcid® and Zantac®. In some embodiments, the subject has GERD-related esophagitis.

Symptoms of GERD are well known in the art. Typic GERD symptoms include, but are not limited to, heartburn (also known as pyrosis), epigastric pain, regurgitation, chest pain, difficulty swallowing (also known as dysphagia), a sour/bitter taste in the mouth, vomiting, bloating, dry cough or throat clearing, sore throat, hoarseness, or laryngitis, shortness of breath, excessive salivation, intolerance of certain foods and liquids and bad breath.

In some embodiments, the herbal composition is administered orally. The unit dose and frequency of administration may vary. In some embodiments, the herbal composition is administered twice a day, once a day, once every two days, once every three days, once every four days or once a week. In some embodiments, the herbal composition is administered for a period of one week, two weeks, three weeks, one month, two months of three months.

The present application is further illustrated by the following examples which should not be construed as limiting. The contents of all references, patents, and published patent applications cited throughout this application are incorporated herein by reference.

### EXAMPLES

### Example 1: The Herbal Supplement of the Present Application is Effective in Alleviating Symptoms of GERD

A trial batch of the herbal supplement of the present application (trial supplement) was prepared by mixing atractloydes extract, dandelion root extract, noni extract and hesperidin at a 9:5:8:2 weight ratio to form a mixture and filling capsules with the mixture. Each capsule contains: 225 Atractloydes macrocephala extract (extraction ratio 10:1), 125 mg dandelion (extraction ratio 10:1), 200 mg noni fruit extract (extraction ration 5:1) and 50 mg hesperidine extract (90% HPLC).

The trial supplement was tried in one 52-year old male with a significant hiatal hernia who had been suffering from severe GERD for over 30 years. In that time span he would respond to each proton pump inhibitor for a period of time then became resistant to the effects. The prescription medications used included omeprazole, lansoprazole, panto prazole, rabeprazole, esomeprazole, and ultimately only responding to a cost prohibitive medication known as dexlansoprazole. An attempt to wean off the medications was tried multiple times over the years only resulting in severe symptoms including not only pyrosis but severe chest and jaw pain. The weaning process was methodical and used protocols of over-the-counter H2 blockers (Pepcid/Zantac) and multiple ‘natural’ remedies. He was ultimately only successful in getting off the prescription proton pump inhibitors after using the above-mentioned herbal combination. He has been off medications for over a year without any side effects.

The above-mentioned herbal combination was also tried on a recalcitrant 51 year old male who was post gastric sleeve, and not responsive to twice daily proton pump inhibitor and nighttime Pepcid. This patient was also successful in weaning off prescription medications in one week at the same dosing regimen described above.

The above description is for the purpose of teaching a person of ordinary skill in the art how to practice the present application. It is not intended to detail all those obvious modifications and variations of it which will become apparent to the skilled worker upon reading the description. It is intended, however, that all such obvious modifications and variations be included within the scope of the present application, which is defined by the following claims. The claims are intended to cover the claimed components and steps in any sequence effective to meet the objectives there intended, unless the context specifically indicates the contrary.

### Example 2: The Herbal Supplement of the Present Application is Effective in Alleviating Symptoms of GERD in an Expanded Study

In an expanded observational study, 31 participants aged 24-67 (median age 47.5) took two capsules (each capsule contains: 225 Atractloydes macrocephala extract (extraction ratio 10:1), 125 mg dandelion (extraction ratio 10:1), 200 mg noni fruit extract (extraction ration 5:1) and 50 mg hesperidine extract (90% HPLC)), twice daily for 30 days. The study cohort included 22 females and 9 males, with 14 participants presenting with classic GERD pyrosis, 7 with mid-epigastric pain only, and 10 with a combination of GERD pyrosis and epigastric pain. Notably, 21 of the participants were on daily proton pump inhibitors (PPIs), while 10 were not on any medications. Among the cohort, 9 had gastrointestinal comorbidities such as inflammatory bowel disease (IBD), small intestinal bacterial overgrowth (SIBO), and morbid obesity, and 7 had hiatal hernias ranging from 2 cm to 4 cm. All participants underwent endoscopy, revealing visual esophagitis in 17 cases and pathologic documentation of esophagitis in 31 cases, including 1 case diagnosed with Barrett's esophagus.

Following the 30-day regimen, of those who were on daily PPI, 17 participants successfully discontinued all medications, 2 reduced PPI use by 75%, 1 transitioned to daily H2 blockers only, and 1 remained on PPIs due to Barrett's esophagus. Additionally, all 31 participants who reported having bloating and pain found relief.

The product of the present application demonstrated a high level of patient satisfaction, as evidenced by 14 unsolicited requests for additional doses of the product, with 4 participants consistently returning monthly for more than two bottles. There were no reported side effects. These findings suggest that the natural GERD product of the present application may offer a significant therapeutic benefit, particularly in reducing reliance on conventional GERD medications while alleviating symptoms and improving overall gastrointestinal health.

What is claimed is:

1. An herbal supplement, comprising two or more herbal components selected from the group consisting of atractloydes extract, dandelion root extract, noni extract, hesperidin extract and quebracho extract, wherein the supplement is formulated for oral administration for alleviation of symptoms of gastroesophageal reflux disease.

2. The herbal supplement of claim 1, comprising three or more components selected from the group consisting of atractloydes extract, dandelion root extract, noni extract, hesperidin extract and quebracho extract.

3. The herbal supplement of claim 2, comprising four or more components selected from the group consisting of atractloydes extract, dandelion root extract, noni extract, hesperidin extract and quebracho extract.

4. The herbal supplement of claim 3, comprising:
atractloydes extract;
dandelion root extract;
noni extract; and
hesperidin extract.

5. The herbal supplement of claim 3, comprising:
atractloydes extract;
dandelion root extract;
noni extract;
hesperidin extract and
quebracho extract.

6. The herbal supplement of claim 2, wherein the herbal supplement is formulated in a unit dosage form and wherein each unit dose comprises at least three components selected from the group consisting of:
atractloydes extract in the range of 45-1125 mg;
dandelion root extract in the range of 25-625 mg;
noni fruit extract in the range of 40-1000 mg; and
hesperidin extract in the range of 10-250 mg

7. The herbal supplement of claim 6, wherein the atractloydes extract is Atroctloydes macrocephala extract having an extraction ratio of 10:1, wherein the dandelion root extract has an extraction ratio of 10:1, wherein the noni fruit extract has an extraction ratio of 5:1 and wherein the hesperidin extract has a purity of 90% HPLC.

8. The herbal supplement of claim 7, wherein each unit dosage comprises:
atractloydes extract in the range of 100-450 mg;
dandelion root extract in the range of 50-250 mg,
noni fruit extract in the range of 80-400 mg, and
hesperidin extract in the range of 20-100 mg.

9. The herbal supplement of claim 5, wherein the herbal supplement is formulated in a unit dosage form and wherein each unit dose comprises:
red quebracho extract in the range of 50-1250 mg;
atractloydes extract in the range of 50-1250 mg;
dandelion root extract in the range of 300-7500 mg;
noni extract in the range of 100-2500 mg; and
hesperidin in the range of 20-500 mg.

10. The herbal supplement of claim 1, further comprising one or more herbal components selected from the group consisting of citrus unshiu peel extracts, genseng extracts, jujube extracts, deglycyrrbizinated licorice (DGL) and Ceratonia sibqua (Carob) extracts.

11. A method for alleviating symptoms of a gastroesophageal reflux disease (GRED) in a human subject, comprising:
orally administering to the human subject in need thereof an effective amount of the herbal supplement of claim 1.

12. The method of claim 11, wherein the herbal supplement is administered daily for a period of one week or longer.

13. The method of claim 11, wherein the herbal supplement is administered twice a day for a period of one week or longer.

14. The method of claim 11, wherein the herbal supplement is administered twice a day and wherein each administration contains Atroctloydes macrocephala extract (extraction ration 10:1) 450 mg, dandelion root extract (extraction ration 10:1) 250 mg, noni fruit extract (extraction ratio 5:1) 400 mg, and hesperidin extract (90% HPLC) 100 mg.

15. The method of claim 11, wherein symptoms of GRED are selected from the group consisting of heartburn, epigastric pain, regurgitation, chest pain, difficulty swallowing, a sour/bitter taste in the mouth, vomiting, bloating, dry cough and throat clearing, sore throat, hoarseness, laryngitis, shortness of breath, excessive salivation, and bad breath.

16. The method of claim 11, wherein the subject is diagnosed with GRED.

17. The method of claim 11, wherein the subject has been treated with proton pump inhibitors and/or H2 blockers.

18. A method for preparing the herbal supplement of claim 1, comprising:
mixing the herbal components to form a mixture; and
formulating the mixture into unit doses suitable for oral administration.

19. The method of claim 18, wherein the unit doses are in capsule form.

20. The method of claim 18, wherein the unit doses are in tablet form.
